(19) United States
(12) Patent Application Publication
(10) Pub. No.: US 2015/0044281 A1
(43) Pub. Date: Feb. 12, 2015

(54) METHODS FOR THE TREATMENT OF MULTIPLE SCLEROSIS AND OTHER DEMYELINATING DISORDERS

(71) Applicant: THE REGENTS OF THE UNIVERSITY OF COLORADO, A BODY CORPORATE, Denver, CO (US)

(72) Inventors: Linda R. Watkins, Boulder, CO (US); Lisa Loram, Denver, CO (US); Melissa J. Mahoney, Boulder, CO (US)

(21) Appl. No.: 14/370,724

(22) PCT Filed: Jan. 7, 2013
(86) PCT No.: PCT/US13/20542

§ 371 Date: Jul. 3, 2014

Related U.S. Application Data
(60) Provisional application No. 61/583,793, filed on Jan. 6, 2012.

(51) Int. Cl. A61K 9/50; A61K 31/713

(52) U.S. Cl. CPC A61K 9/5031; A61K 31/713
USPC 424/451; 514/44 R

(57) ABSTRACT

This invention provides compositions and methods for preventing multiple sclerosis or other demyelinating disorders, inhibiting multiple sclerosis or other demyelinating disorders, or prolonging remission from MS or other demyelinating disorders comprising administering to a subject a microparticle comprising a biodegradable material comprising at least one IL-10 coding sequence.

### CROSS-REFERENCE TO RELATED APPLICATIONS

This application claims the benefit of U.S. Provisional Patent Application No. 61/583,793, filed Jan. 6, 2012 and is incorporated herein by reference.

### STATEMENT REGARDING FEDERALLY FUNDED RESEARCH

This invention was made with government support under grant numbers DE021966, DE020247, DA024044 awarded by the National Institute of Health. The government has certain rights in the invention

### FIELD OF THE INVENTION

This invention relates to treating clinical conditions associated with multiple sclerosis and other demyelinating diseases and symptoms associated therewith by administering microparticles comprising one or more anti-inflammatory cytokine coding sequences to a subject in need of such a treatment.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under applicable statutory provisions.

Multiple sclerosis (MS) is a complex neurological disease characterized by deterioration of central nervous system (CNS) myelin. Myelin is an insulating material composed primarily of lipids that protects nerve fibers—axons—that transmit electric impulses throughout the body. Demyelination of axons in MS results in axon degeneration and neuronal cell death, but more specifically MS destroys oligodendrocytes, specialized glial cells that generate and maintain myelin.

Oligodendrocyte progenitors are generated in ventral areas of the developing brain from a glial progenitor. Oligodendrocyte progenitors actively migrate and proliferate, populating the CNS, eventually maturing to target and extend myelin sheaths along the axons. However a subpopulation of the oligodendrocyte progenitors remains as resident, undifferentiated cells to play a role if myelin is damaged or deteriorates. People with MS suffer attacks when T-cells cross the blood brain barrier and attack the myelin sheath that coats axons of the CNS causing inflammation.

Current treatments for MS remain suboptimal and often require daily subcutaneous injections leaving substantial irritation at the injection sites. Identifying treatments that would require less frequent treatments would impact significantly the quality of life for MS patients and patients with other demyelinating disorders. However, despite substantial research into and development of therapies for multiple sclerosis and other demyelinating disorders, there is still a large unmet need for safe, effective, and easy-to-administer treatments.

### SUMMARY OF THE INVENTION

Immune cells both in the peripheral and the central nervous system contribute significantly to development and recurrence of multiple sclerosis and other demyelinating disorders in animal models and in humans. These immune cells produce a pro-inflammatory microenvironment contributing to neural hyperexcitability and pain, and also to neurodegeneration. The present invention provides methods for treating multiple sclerosis and other demyelinating disorders, symptoms associated with multiple sclerosis and other demyelinating disorders, and prolonging remission and preventing relapse of symptoms and disease progression by administering to a subject, typically intrathecally, a microparticle comprising a plasmid DNA (pDNA). The pDNA comprises a bacterial backbone driving the expression of at least one interleukin-10 (IL-10) coding sequence along with, optionally, one or more additional anti-inflammatory cytokine coding sequences. The pDNA is encapsulated in biodegradable microparticles, and in most embodiments, the microparticles are suspended in a diluent to form a therapeutic composition. The present inventors have discovered that the therapeutic microparticle composition was found to extend the length of time of cytokine delivery to the central nervous system in vivo far beyond viral vector delivery models and for up to three weeks beyond the length of time achieved using a bacterial vector-driven anti-inflammatory cytokine coding sequence when delivered as naked DNA. Moreover, in some cases this lengthening of efficacy was achieved using 15× less than the amount of plasmid DNA to achieve the same result with the bacterial vector delivered as naked DNA. Optionally, the pDNA also comprises at least one nuclear targeting sequence.

Thus, some embodiments of the invention provide methods for treatment of multiple sclerosis or other demyelinating diseases or disorders or symptoms associated therewith in a subject comprising intrathecally administering to the subject a therapeutic microparticle composition comprising: plasmid DNA comprising a bacterial backbone and at least one IL-10 coding sequence along with, optionally, one or more additional anti-inflammatory cytokine coding sequences; microparticles encapsulating the plasmid DNA, where the microparticles degrade to deliver plasmid DNA over a period of greater than 40 days; and a diluent; where the therapeutic microparticle composition provides a therapeutically effective dose at about 0.010 μg pDNA per gram animal weight to about 0.90 μg pDNA per gram animal weight.

Yet other embodiments of the present invention provide methods for preventing relapse of multiple sclerosis or other demyelinating disease or disorder or sustaining remission in a subject comprising administering to the subject a therapeutic microparticle composition comprising: plasmid DNA comprising a bacterial backbone and at least one IL-10 coding sequence along with, optionally, one or more additional anti-inflammatory cytokine coding sequences; microparticles encapsulating the plasmid DNA, where the microparticles degrade to deliver plasmid DNA over a period of greater than 40 days; and a diluent; wherein the therapeutic microparticle composition provides a therapeutically effective dose at about 0.010 μg pDNA per gram animal weight to about 0.90 μg pDNA per gram animal weight.

Optionally in the embodiments described, the plasmid DNA comprises at least one nuclear targeting sequence located either 5′ to the at least one IL-10 coding sequence or 3′ to the at least one IL-10 coding sequence. In other aspects of the present invention, the plasmid DNA comprises two nuclear targeting sequences where one nuclear targeting sequence is positioned 5′ of the at least one IL-10 coding sequence, and one nuclear targeting sequence is positioned 3′ of the at least one coding sequence.

Preferably, the therapeutic composition is administered intrathecally.

In some aspects of the present invention, the microparticles comprise one or more of poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polycaprolactone, poly-3-hydroxybutyrate or polyorthoesters. In certain aspects of the present invention, the microparticles comprise PLGA. In other aspects of the invention, the microparticles comprise a mixture of biodegradable polymers representing different, complimentary release profiles.

In certain embodiments of the methods, the therapeutic microparticle composition is delivered approximately every 40 to 80 days as needed for therapeutic effect for therapeutic effect, e.g., up to one year. In other embodiments, the therapeutic microparticle composition is delivered approximately every 40 to 80 days as needed for therapeutic effect for greater than one year. In yet other embodiments, the therapeutic microparticle composition is delivered as needed for therapeutic effect approximately every 40 to 80 days for the life of the subject.

The methods of the present invention also may be employed as a research tool to identify pharmaceuticals, small molecules and/or biologics that may be used in conjunction in a “cocktail” with the therapeutic microparticle compositions.

### BRIEF DESCRIPTION OF THE DRAWINGS

FIG. 1 is a graph plotting motor score versus time post-onset of motor symptoms (in days) in treated and untreated experimental autoimmune encephalomyelitis (EAE) model rats.

FIG. 2 is a survival curve plotting percent survival versus time post-onset of motor symptoms (in days) in treated and untreated EAE model rats.

FIG. 3 is a graph plotting voluntary running activity versus time post-onset of motor symptoms (in days) in treated and untreated EAE model rats.

### DETAILED DESCRIPTION OF THE INVENTION

### Definitions

Unless defined otherwise, all technical and scientific terms used herein have the same meaning as commonly understood by one of ordinary skill in the art to which this invention belongs. All publications, published patent applications and patents mentioned herein are incorporated by reference in their entirety for the purpose of describing and disclosing devices, animal models, formulations and methodologies that may be used in connection with the presently described invention.

The term “anti-inflammatory cytokine” as used herein refers to a protein that decreases the action or production of one or more proinflammatory cytokines or proteins produced by nerves, neurons, glial cells, endothelial cells, fibroblasts, muscle, immune cells or other cell types. Such inflammatory cytokines and proteins include, without limitation, interleukin-1 beta (IL-1β), tumor necrosis factor-alpha (TNF-a), interleukin-6 (IL-6), inducible nitric oxide synthetase (iNOS) and the like. Non-limiting examples of anti-inflammatory cytokines include interleukin-10 (IL-10) including viral IL-10, interleukin-4 (IL-4), interleukin-13 (IL-13), alpha-MSH, transforming growth factor-beta 1 (TGFβ1), and the like. Thus, the full-length molecules and fragments of anti-inflammatory cytokines, as well as anti-inflammatory cytokines with modifications, such as deletions, additions and substitutions (either conservative or non-conservative in nature), to the native sequence, are intended for use herein, so long as the anti-inflammatory cytokine is therapeutically effective. Modifications may be deliberate, as through site-directed mutagenesis, or may be accidental, such as through mutations of hosts which produce the proteins or errors due to PCR amplification. Accordingly, active proteins are typically substantially homologous to the parent sequence, e.g., proteins are typically about 70 . . . 80 . . . 85 . . . 90 . . . 95 . . . 98 . . . 99%, etc. homologous to the parent sequence.

The term DNA “control sequences” refers collectively to promoter sequences, polyadenylation signals, transcription termination sequences, upstream regulatory domains, origins of replication, internal ribosome entry sites, enhancers, and the like, which collectively provide for the replication, transcription and translation of a coding sequence in a recipient cell. Not all of these types of control sequences need to be present so long as the selected coding sequence is capable of being replicated, transcribed and translated in an appropriate host cell.

A “coding sequence” of an anti-inflammatory cytokine or a sequence that “encodes” an anti-inflammatory cytokine is a nucleic acid molecule that is transcribed (in the case of DNA) and translated (in the case of mRNA) into a polypeptide in vivo when placed under the control of appropriate control sequences. The boundaries of the coding sequence are determined by nucleotides corresponding to a start codon at the amino terminus and nucleotides corresponding to a translation stop codon at the carboxy terminus.

The terms “effective amount” or “therapeutically effective amount” of a therapeutic microparticle composition used in the methods of the invention refer to a nontoxic but sufficient amount of the therapeutic microparticle composition to provide the desired response, such as a reversal of damage caused to the nervous system by MS or other demyelinating disorders, relief from symptoms caused by MS or other demyelinating disorders and/or prolonging remission and/or preventing relapse of MS or other demyelinating disorders. The exact amount required will vary from subject to subject, depending on the species, age, and general condition of the subject, the severity of the condition being treated, and the particular plasmid DNA to be delivered, mode of administration, and the like. Dosage parameters for the present methods are provided herein; however, optimization of an appropriate “effective” amount in any individual case may be determined by one of ordinary skill in the art using the methods set forth herein and routine experimentation.

The term “excipient” refers to an inert substance added to a pharmaceutical composition of the invention to further facilitate administration of the therapeutic microparticle composition. Examples, without limitation, of excipients include saline, calcium carbonate, calcium phosphate, various sugars and types of starch, cellulose derivatives, gelatin, hyaluronic acid optionally formulated with a surfactant, Plurnoic F-68, vegetable oils and polyethylene glycols.

The term “glial cells” and “glia” refer to non-neuronal precursor and/or fully-differentiated cells in the nervous system that provide support and nutrition, maintain homeostasis, form myelin, and participate in signal transmission.

By “isolated” when referring to a nucleotide sequence, is meant that the indicated molecule is present in the substantial absence of other biological macromolecules of the same type. Thus, an “isolated nucleic acid molecule which encodes a particular polypeptide” refers to a nucleic acid molecule which is substantially free of other nucleic acid molecules that do not encode the subject polypeptide; however, the molecule may include some additional bases or moieties that do not deleteriously affect the basic characteristics of the composition.

The term “nervous system” includes both the central nervous system and the peripheral nervous system. The term “central nervous system” or “CNS” includes all cells and tissue of the brain and spinal cord of a vertebrate. The term “peripheral nervous system” refers to all cells and tissue of the portion of the nervous system outside the brain and spinal cord. Thus, the term “nervous system” includes, but is not limited to, neuronal cells, glial cells, astrocytes, cells in the cerebrospinal fluid (CSF), cells in the interstitial spaces, cells in the protective coverings of the spinal cord, epidural cells (i.e., cells outside of the dura mater), cells in non-neural tissues adjacent to or in contact with or innervated by neural tissue, macrophages, monocytes, cells in the epineurium, perineurium, endoneurium, funiculi, fasciculi, and the like.

The term “nuclear targeting sequence” refers to a nucleic acid sequence which functions to improve the expression efficiency of the anti-inflammatory cytokine in a cell.

“Operably linked” refers to an arrangement of elements where the components so described are configured so as to perform their usual function. Thus, control sequences operably linked to a coding sequence are capable of effecting the expression of the coding sequence. The control sequences need not be contiguous with the coding sequence so long as they function to direct the expression thereof. Thus, for example, intervening untranslated yet transcribed sequences can be present between a promoter sequence and the coding sequence and the promoter sequence can still be considered “operably linked” to the coding sequence.

The term “promoter” is used herein in its ordinary sense to refer to a nucleotide region comprising a DNA regulatory sequence, wherein the regulatory sequence is derived from a gene that is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

For the purpose of describing the relative position of nucleotide sequences in a particular nucleic acid molecule throughout the instant application, such as when a particular nucleotide sequence is described as being situated “upstream,” “downstream,” “3 prime (3′)” or “5 prime (5′)” relative to another sequence, it is to be understood that it is the position of the sequences in the “sense” or “coding” strand of a DNA molecule that is being referred to as is conventional in the art.

The term “research tool” as used herein refers to any methods of the invention using the therapeutic microparticle composition for scientific inquiry, either academic or commercial in nature, including the development of other pharmaceutical and/or biological therapeutics. The research tools of the invention are not intended to be therapeutic or to be subject to regulatory approval; rather, the research tools of the invention are intended to facilitate research and aid in such development activities, including any activities performed with the intention to produce information to support a regulatory submission.

The terms “subject”, “individual” or “patient” may be used interchangeably herein and refer to a vertebrate, preferably a mammal.

The term “therapeutic composition” or “therapeutic microparticle composition” as used herein refers to a composition that has the ability to reverse damage caused to the nervous system by MS or other demyelinating disorder, relieve symptoms caused by MS or other demyelinating disorder and/or prolong remission and/or prevent relapse of MS or other demyelinating disorder as measured in any of the known animal models or by assessment performed in humans including those described herein.

“Treatment” or “treating” MS or other demyelinating disorder includes: (1) preventing MS or other demyelinating disorder, i.e., causing MS or other demyelinating disorder not to develop or to occur with less intensity in a subject that may be predisposed to MS or other demyelinating disorder but does not yet experience or display symptoms, (2) inhibiting MS or other demyelinating disorder, i.e., arresting the development of or reversing symptoms or physiological damage caused by MS or other demyelinating disorder, or (3) prolonging remission from MS or other demyelinating disorder, thereby decreasing relapse of MS.

The practice of the techniques described herein may employ, unless otherwise indicated, conventional techniques and descriptions of organic chemistry, polymer technology, molecular biology (including recombinant techniques), cell biology, biochemistry, and sequencing technology, which are within the skill of those who practice in the art. Specific illustrations of suitable techniques can be had by reference to the examples herein. However, other equivalent conventional procedures can, of course, also be used. Such conventional techniques and descriptions can be found in standard laboratory manuals such as LeDoux (Ed.) (2005), Animal Models of Movement Disorders (Academic Press); Chow, et al., (2008), Using Animal Models in Biomedical Research (World Scientific Publishing Co.); Weir and Blackwell (Eds.), Handbook of Experimental Immunology, Vols. I-IV (Blackwell Scientific Publications); Creighton (1993), Proteins: Structures and Molecular Properties (W.H. Freeman and Company); Sambrook and Russell (2006), Condensed Protocols from Molecular Cloning: A Laboratory Manual; and Sambrook and Russell (2002), Molecular Cloning: A Laboratory Manual (both from Cold Spring Harbor Laboratory Press); Stryer, L. (1995) Biochemistry, Fourth Ed. (W.H. Freeman); Gait (1984), “Oligonucleotide Synthesis: A Practical Approach” (IRL Press); Nelson and Cox (2000), Lehninger, Principles of Biochemistry, Third Ed. (W. H. Freeman); and Berg et al. (2002) Biochemistry, Fifth Ed. (W.H. Freeman); all of which are herein incorporated in their entirety by reference for all purposes.

Note that as used herein and in the appended claims, the singular forms “a,” “an,” and “the” include plural referents unless the context clearly dictates otherwise.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

### Methods of the Invention

Effective conduction of action potentials in the mammalian central nervous system (CNS) requires proper ensheathment and insulation of neuron axons by myelin. Impairments of oligodendrocyte cells, the myelinogenic cells of the mammalian CNS, cause a number of debilitating and often fatal human conditions. The incapacitating effects of myelin defects are typified by motor and sometimes cognitive deficiencies and are readily apparent in congenital dysmyelinating disorders as well as acquired demyelinating conditions such as multiple sclerosis and cerebral palsy. Immune cells both in the peripheral and the central nervous system contribute significantly to development and recurrence of multiple sclerosis and other demyelinating disorders in animal models and in humans. These immune cells produce a pro-inflammatory microenvironment contributing to neural hyperexcitability and pain, and also to neurodegeneration.

Multiple sclerosis (MS), a progressive, neurodegenerative disease of the CNS, occurs most often in a relapsing/remitting form, in which a period of demyelination is followed by a period of functional recovery (Weiner, Ann Neurol, 65:239-248 (2009)). The recovery stage involves remyelination via migration and maturation of oligodendrocyte precursor cells or oligodendrocyte progenitor cells (OPCs) (Chari, Int Rev Neurobiol, 79:589-620 (2007)). However, as the disease progresses, remyelination fails with progressive loss of function (Blakemore and Keirstead, J Neuroimmunol, 98:69-76 (1999)). Possible explanations for remyelination failure of intact axons include defects in OPC recruitment to the site of demyelination or defects in OPC differentiation into myelinating oligodendrocytes. Although studies indicate that both aspects of OPC biology are altered in MS, the molecular mechanisms that orchestrate these processes within the adult CNS are incompletely understood but are known to include inflammation.

Other conditions mediated by a loss of myelin include an ischemic demyelination condition, an inflammatory demyelination condition, a pediatric leukodystrophy, mucopolysaccharidosis, perinatal germinal matrix hemorrhage, cerebral palsy, periventricular leukoinalacia, radiation-induced conditions, and subcortical leukoencephalopathy due to various etiologies, and mental illnesses, such as schizophrenia. Ischemic demyelination conditions include cortical stroke, Lacunar infarct, post-hypoxic leukoencephalopathy, diabetic leukoencephalopathy, and hypertensive leukoencephalopathy. Inflammatory demyelination conditions include multiple sclerosis, Schilder's Disease, transverse myelitis, optic neuritis, post-vaccination encephalomyelitis, and post-infectious encephalomyelitis. Pediatric leukodystrophy conditions include lysosomal storage diseases (e.g., Tay-Sachs Disease), Cavavan's Disease, Pelizaeus-Merzbacher Disease, and Crabbe's Globoid body leukodystrophy. An example of mucopolysaccharidosis is Sly's Disease. Radiation-induced conditions include radiation-induced leukoencephalopathy and radiation induced myelitis. Etiologies causing subcortical leukoencephalopathy include HIV/AIDS, head trauma, and multi-infarct states.

The invention provides methods for treating MS or other demyelinating or dysmyelinating disorder and symptoms and physiological damage associated with MS or other demyelinating or dysmyelinating disorder. The invention also provides for using the methods of the invention in research of demyelinating states, including identifying pharmaceuticals, small molecules and/or biologics that may be used in conjunction in a “cocktail” with the therapeutic microparticle compositions. The methods comprise the step of administering to a subject, preferably at least intrathecally, a plasmid DNA (pDNA) comprising a bacterial backbone and at least one IL-10 coding sequence along with, optionally, one or more additional anti-inflammatory cytokine coding sequences, where the pDNA is encapsulated in biodegradable microparticles. The microparticles are generally suspended in a diluent to from a therapeutic composition. Optionally, the pDNA may also comprise at least one nuclear targeting sequence where the at least one nuclear targeting sequence is 5′, 3′ or both to the at least one IL-10 coding sequence, and, optionally, to one or more additional anti-inflammatory cytokine coding sequences. The therapeutic microparticle composition was found to extend the length of time of anti-inflammatory cytokine delivery in vivo far beyond viral vector delivery models and for up to three weeks beyond the length of time achieved using a bacterial vector driven anti-inflammatory cytokine coding sequence when delivered as naked DNA. Moreover, this lengthening of efficacy was achieved using less than 15× less plasmid DNA to achieve the same result with the bacterial vector delivered as naked DNA. In addition, it has been found that the therapeutic microparticle compositions of the invention can be administered multiple times without any significant immunological reaction and without inducing any significant dose tolerance.

The plasmid DNA (pDNA) used in the methods of the present invention comprises a bacterial backbone, at least one IL-10 coding sequence, at least one nuclear targeting sequence 5′ (upstream), 3′ (downstream) or both of the at least one IL-10 coding sequence, and one or more DNA control sequences. Optionally, the pDNA can also comprise one or more additional anti-inflammatory cytokine coding sequences, and/or a marker sequence to allow for selection of transformed cells during amplification of the pDNA. The bacterial backbone can be any bacterial backbone known to those with skill in the art. Backbones typically selected are those that, e.g., contain or lack appropriate restriction sites to allow ease of cloning, may be produced and isolated with ease, are not immunogenic, and the like. For example, bacterial backbones derived from E. coli are of use in the present invention.

The plasmid DNA comprises at least one IL-10 coding sequence. IL-10 may be used in wildtype form, or the IL-10 may be a mutant IL-10. One mutant IL-10 of interest contains one or more mutations that cause amino acid substitutions, additions or deletions as compared to wildtype IL-10 in the “hinge” region of the IL-10 protein. The human IL-10 protein is a homodimer, where each monomer comprises six alpha helices A→F, the length of which are 21, 8, 19, 20, 12 and 23 amino acids, respectively. Helices A→D of one monomer noncovalently interact with helices E and F of a second monomer, forming a noncovalent V-shaped homodimer. The “hinge” region targeted for mutation according to the present invention comprises the amino acids between the D and E alpha helices on one or both monomers at approximately amino acid position X to position Y of wildtype IL-10. For example, mutant rat and human IL-10 proteins have been described in which the phenylalanine at position 129 of the wildtype sequence has been replaced with a serine residue. (See, e.g., Sommer, et al., WO2006/130580 and Milligan, et al., Pain, 126:294-308 (2006).) The resulting mutant IL-10 is referred to as IL-10^{F129S}. Other substitutions for the wildtype phenylalanine at amino acid position 129 may be, e.g., threonine, alanine, or cysteine. Thus the present invention in yet another aspect encompasses one or more substitutions at amino acid position 129 or at other amino acids within the hinge region of the IL-10 protein.

Additional anti-inflammatory cytokines of use in the present invention include but are not limited to interleukin-4 (IL-4), interleukin-13 (IL-13), alpha-MSH, transforming growth factor-beta 1 (TGFβ1), and the like.

Nuclear targeting sequences of the present invention are sequences that promote expression of the protein(s) encoded by the at least one IL-10 coding sequence and the optional, additional anti-inflammatory cytokine coding sequence(s). For example, in one aspect the nuclear targeting sequences may bind to nuclear transport chaperone proteins, facilitating uptake of the plasmid DNA by the cell nucleus. Such sequences include but are not limited to interspersed (or dispersed) DNA repeats or repetitive sequences such as transposable elements, flanking or terminal repeats such as the long terminal repeats (LTRs) on retrovirus genomes such as SV40s, tandem repeats, and the inverted terminal repeats (ITRs) of viral genomes such as Adeno-Associated Virus and Adenovirus. In other aspects, the nuclear targeting sequences are sequences that act to bind transcription factors for import into the nucleus, such as enhancer sequences.

In addition to a bacterial backbone, at least one IL-10 coding sequence and optional one or more additional anti-inflammatory cytokine coding sequences and, optionally, one or more nuclear targeting sequences, the plasmid DNA of the present invention comprises one or more DNA control sequences, such as promoter sequences, polyadenylation signals, transcription termination sequences, upstream regulatory domains, origins of replication, internal ribosome entry sites and the like, which collectively provide for the replication, transcription and translation of the anti-inflammatory cytokine coding sequence(s) in a recipient cell. Not all of these control sequences need always be present so long as the anti-inflammatory cytokine coding sequences are capable of being replicated, transcribed and translated in an appropriate host cell. Promoter sequences of use in the present invention include but are not limited to chicken or human β-actin promoters, cytomegalovirus immediate early promoters, glyceraldehydes 3-phosphate dehydrogenase (GADPH) promoters, elongation factor 1α (eF1α) promoter, GFAP promoter, murine leukemia virus (MLV) promoter, herpes simples virus thymidine kinase (TK) promoter, and woodchuck hepatitis virus post-transcriptional regulatory element (WPRE) promoters; upstream regulatory domains of use in the present invention include but are not limited to cytomegalovirus immediate early promoter enhancers, mouse mammary tumor virus (MMTV) enhancer and simian virus 40 (SV40) enhancer; and polyadenylation signals of interest in the present invention include but are not limited to SV40 polyadenylation signal, bovine growth hormone polyadenylation signal, and synthetic polyadenylation signals. Optionally, the plasmid DNA of the present invention will also comprise a selection marker gene, such as that coding for antibiotic resistance. Marker genes of use in the present invention include but are not limited to neomycin, hygromycin-B, ampicillin, kanomycin, or puromycin.

For example, plasmids comprising the rat IL-10 sequence flanked by two AAV ITRs, a cytomegalovirus immediate early promoter enhancer, a chicken β-actin promoter, a polyadenylation signal, and a herpes simplex thymidine kinase promoter driving a neomycin resistance marker were used in some experiments demonstrating the usefulness of the present invention. In other experiments, plasmids comprising the human IL-10 sequence flanked by two AAV ITRs, a cytomegalovirus immediate early promoter enhancer, a cytomegalovirus immediate early promoter, a polyadenylation signal, and an ampicillin resistance marker were used. Details of these plasmids are disclosed in Milligan, et al., Pain 126:294-308 (2006).

Once the pDNA has been constructed, amplified and isolated by techniques known in the art, the pDNA is then encapsulated within microparticles. Techniques for encapsulating pDNA vary depending on the type of microparticles used and such techniques are described in more detail infra. The microparticles of the present invention may be comprised of any biodegradable polymer. To be used successfully as a biodegradable polymer in the controlled drug delivery formulations of the present invention, the material must be chemically inert and free of leachable impurities. Ideally the polymer also has an appropriate physical structure, with minimal undesired aging, and is readily processable. Some of the materials include poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), and poly(methacrylic acid). Biodegradable polymers of particular use in the present invention include polylactides (PLA), polyglycolides (PGA), poly(lactide-co-glycolides) (PLGA), polyanhydrides, polycaprolactone, poly-3-hydroxybutyrate and polyorthoesters. Such biodegradable polymers have been characterized extensively and can be formulated to exhibit desired degradation properties as is known in the art (see, e.g., Edlund & Albertsson, Degradable Aliphatic Polyesters, pp. 67-112 (2002), Barman, et al., J. of Controlled Release, 69:337-344 (2000); Cohen, et al., Pharmaceutical Res., (8): 713-720 (1991)).

In one particular embodiment of the invention, the polymer comprises poly(lactide-co-glycolides) (PLGA). PLGA is a copolymer which is used in a host of FDA approved therapeutic devices, owing to its biodegradability and biocompatibility. PLGA is synthesized by means of random ring-opening co-polymerization of two different monomers, the cyclic dimers (1,4-dioxane-2,5-diones) of glycolic acid and lactic acid. Common catalysts used in the preparation of this polymer include tin(II) 2-ethylhexanoate, tin(II) alkoxides, or aluminum isopropoxide. During polymerization, successive monomeric units of glycolic or lactic acid are linked together in PLGA by ester linkages, thus yielding a linear, aliphatic polyester as a product.

Depending on the ratio of lactide to glycolide used for the polymerization, different forms of PLGA can be obtained: these are usually identified in regard to the monomers' ratio used (e.g., PLGA 75:25 identifies a copolymer whose composition is 75% lactic acid and 25% glycolic acid). PLGA degrades by hydrolysis of its ester linkages in the presence of water. It has been shown that the time required for degradation of PLGA is related to the monomers' ratio used in production: the higher the content of glycolide units, the lower the time required for degradation. An exception to this rule is the copolymer with 50:50 monomers' ratio which exhibits the faster degradation (about two months). In addition, polymers that are end-capped with esters (as opposed to the free carboxylic acid) demonstrate longer degradation half-lives. Of particular use in the present invention is PLGA having a composition of between 20% and 80% lactic acid and between 80% and 20% glycolic acid. More preferred for use in the present invention is PLGA having a composition of between 65% and 35% lactic acid and between 35% and 65% glycolic acid. In one aspect of the present invention, PLGA having a composition of 50% lactic acid and 50% glycolic acid is used.

Additionally, the pDNA may be encapsulated in batches of microparticles having different release profiles; for example, 10% of the pDNA to be delivered may be encapsulated in microparticles having, e.g., a one day to four week release profile; 30% of the pDNA to be delivered may be encapsulated in microparticles having, e.g., a three week to six week release profile; 30% of the pDNA to be delivered may be encapsulated in microparticles having, e.g., a six week to ten week release profile; and 30% of the pDNA to be delivered may be encapsulated in microparticles having, e.g., an eight week to twelve week release profile. In such an embodiment, a single type of biodegradable polymer may be used, but used in formulations with different release profiles; alternatively, different biodegradable polymers having different release characteristics may be used. In yet another embodiment, the formulation of the microparticles may be varied so as to change the surface of the microparticles to enhance or retard, as desired, the travel of the therapeutic composition through, e.g., the spinal column.

Once microparticles are obtained, they are suspended in an acceptable diluent to form a therapeutic composition for administration to an animal. Such diluents (or excipients) include any pharmaceutical agent that does not itself induce the production of antibodies harmful to the individual receiving the composition, and that may be administered without undue toxicity. Pharmaceutically acceptable diluents may comprise sorbitol, alum, dextran, sulfate, large polymeric anions, any of the various TWEEN compounds, and liquids such as water, saline, glycerol or ethanol, oil and water emulsions, or adjuvants such as Freund's adjuvant. Pharmaceutically acceptable salts can be included therein as well, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like may be present in such vehicles. In one aspect of the invention, different diluents are used based on their ability to migrate through the targeted site in the nervous system, such as, e.g., spinal column. For example, for intrathecal delivery in an adult human, diluents may be preferred that favor more rapid spread of the therapeutic composition through the length of the spinal column; conversely, in children or small animals where the length of the spinal column is less of a concern, a diluent may be used that does not disperse the therapeutic composition quickly. A thorough discussion of pharmaceutically acceptable diluents/excipients is available in Remington: The Science and Practice of Pharmacy, 21^{st} ed., Lippincott Williams & Wilkins (2005). Preferred diluents include but are not limited to Physiosol®, artificial cerebrospinal fluid, preservative-free 0.9% NaCl, lactated Ringer's injection solution, and Elliotts B® Solution.

Synthesizing and administering the compositions to be used in the methods of the present invention involve a series of steps. First, a plasmid is constructed comprising the various components described supra. Then the plasmid DNA (pDNA) is amplified and isolated by techniques well known in the art. Once the pDNA is isolated, it will be combined with polymers to form pDNA-containing microparticles. Methods for microparticle formation vary depending on the polymers used, however, a double emulsion technique is typically employed. First, a polymer is dissolved in an organic solvent. Next, pDNA is suspended in an aqueous solution and is added to the polymer solution. The two solutions are then mixed to form a first emulsion. The solutions can be mixed by vortexing or shaking or by passage through a particulate medium producing turbulence, or the mixture can be sonicated. Most preferable is any method by which the nucleic acid receives the least amount of damage in the form of nicking, shearing, or degradation, while still allowing the formation of an appropriate emulsion. During this process, the polymer forms into microparticles, many of which contain pDNA. If desired, one can isolate a small amount of the nucleic acid at this point in order to assess integrity, e.g., by gel electrophoresis.

The first emulsion is then added to an organic solution. The solution can be comprised of, for example, methylene chloride, ethyl acetate, or acetone, typically containing polyvinyl alcohol (PVA), and often having approximately a 1:100 ratio of the weight of PVA to the volume of the solution. The first emulsion is generally added to the organic solution with stirring in a homogenizer or sonicator. This process forms a second emulsion which is subsequently added to another organic solution with stirring (e.g., in a homogenizer). In one aspect of this method, the latter solution is 0.05% w/v PVA. The resultant microparticles are washed several times with water to remove the organic compounds. In some aspects of the present invention, more than approximately 40% of the resulting microparticles contain pDNA. In yet other aspects, more than approximately 50% of the resulting microparticles contain pDNA, in yet other aspects of the present invention, more than 55% of the resultant microparticles contain pDNA.

In one embodiment, microparticles were formulated for use in EAE rat experiments where 16-18 μg of pDNA was used initially to form the microparticles, and 8 μg of pDNA was ultimately incorporated into the microparticles per milligram PLGA material. The 8-10 μg that was not incorporated into microparticles was recovered to be used in future microparticle preparation. The present invention provides a significant savings over the prior art in terms of pDNA that must be synthesized, isolated and delivered to achieve therapeutic effect.

The ability to internalize differently-sized microparticles varies with cell type. In certain embodiments of the invention, macrophages and antigen-presenting cells were targeted. Such cells more efficiently internalize microparticles of less than about 5μ (see Shakweh, et al., Eur J of Pharmaceutics and Biopharmaceutics 61(1-2):1-13 (2005)). Thus, if desired, particles may be passed through sizing screens to selectively remove those larger than the desired size. In one particular aspect of the invention, microparticles of less than 5μ are used in the therapeutic composition, and in other particular aspects of the invention, microparticles of less than 3μ are used in the therapeutic composition. After washing, the particles can either be used immediately or be lyophilized for storage. The size distribution of the microparticles prepared by the methods described herein can be determined with, e.g, a Coulter™ counter or laser diffraction. Alternatively, the average size of the particles can be determined by visualization under a microscope fitted with a sizing slide or eyepiece. Alternatively, a scanning electron microscope can be used to assess both size and microparticle morphology.

Once pDNA-containing microparticles are obtained, the microparticles can be suspended immediately in diluent or lyophilized for storage. The combination of the microparticles and diluent forms the therapeutic microparticle composition that can be administered intrathecally to an animal subject. The recombinant plasmid DNA vectors can be introduced either in vivo or in vitro (also termed ex vivo) to treat preexisting neuronal damage, neuropathies and MS and other demyelinating disorders or symptoms thereof. If transduced in vitro, the desired recipient cell or spinal fluid is removed from the subject, treated with pDNA-containing microparticles and reintroduced into the subject. Alternatively, syngeneic or xenogeneic cells can be transformed for delivery where such cells typically do not generate an inappropriate immune response in the subject. Additionally, neural progenitor cells can be treated in vitro and then delivered to the central nervous system. Alternatively, recombinant plasmids, or cells treated with plasmid in vitro, can be delivered directly by intrathecal delivery. An advantage of intrathecal gene therapy is that the intrathecal route mimics lumbar puncture administration (i.e., spinal tap) already in routine use in humans.

Because the therapeutic composition of the invention does not significantly induce an immune response or dose tolerance in subjects, it can be administered as needed for therapeutic effect. That is, the therapeutic composition can be delivered approximately every 40 to 80 days (or as required according to the degradation profile of the biodegradable polymer) as needed for therapeutic effect for shorter-term therapy. However, when longer-term therapy is desired, the therapeutic composition can be delivered approximately every 40 to 80 days as needed for therapeutic effect for greater than one year; and if necessary, for the life of the subject.

Dosage ranges of the therapeutic microparticle compositions used in the methods of the present invention vary from subject to subject, depending on the species, age, and general condition of the subject, the severity of the condition being treated, and the particular plasmid DNA to be delivered, mode of administration, and the like. Dosage ranges include a therapeutically effective dose at about 0.010 μg pDNA per gram animal weight to about 0.90 μg pDNA per gram animal weight, or about 0.04 μg pDNA per gram animal weight to about 0.70 μg pDNA per gram animal weight, or about 0.08 μg pDNA per gram animal weight to about 0.50 μg pDNA per gram animal weight.

The microparticles used in the methods of the present invention may be co-administered in a “cocktail” with other therapeutic agents useful in treating MS or other demyelinating disorders, such as gangliosides; antibiotics, neurotransmitters, neurohormones, toxins, neurite promoting molecules; or antimetabolites and precursors of neurotransmitter molecules. Additionally, the microparticles used in the methods of the present invention may be co-administered with cells, such as glial precursor cells or other stem cells, including bioengineered cells.

Generally, any method known in the art can be used to monitor success of treatment in humans, including both clinical and phenotypic indicators. For example, MRI can be used for visualizing brain white matter and studying the burden of demyelinating lesions as currently practiced for monitoring MS patients. Magnetic resonance spectroscopy measurement of N-acetyl-aspartate levels can be used to assess impact on local neuron/axon survival by using paramagnetic particles to label cells before transplantation, enabling cell dispersion to be tracked by MRI. Alternatively or in addition, magnetization transfer contrast can be used to monitor remyelination (Deloire-Grassin, J. Neurol. Sci., 178:10-16 (2000)). Serial neurophysiology monitoring techniques can also be used to assess improvement over time. Additionally, electrophysiological measures of sensory and motor nerve conductivity, for example H-wave response, are classical methods used for monitoring neuropathies linked to demyelinating peripheral lesions (Lazzarini et al, Eds (2004) Myelin Biology and Disorders (Elsevier Academic Press)). Other approaches to more generalized phenotypic neurophysiological assessment are described in Leocani et al., Neurol Sci., 21(4 Suppl 2):S889-91 (2000), which may be useful for interventions aimed at multifocal or more diffuse myelin repair. For example, demyelination causes alterations of stature (trembling, shivering) and locomotion, and children with leukodystrophies have motor and intellectual retardation Improvement in these states may be assessed to monitor therapeutic success.

A variety of animal models have been developed for the study of MS and other demyelinating disorders; for example, an animal model of experimental autoimmune encephalomyelitis (EAE)—also known as experimental allergic encephalomyelitis—has been developed to study brain inflammation. EAE is an inflammatory demyelinating disease of the central nervous system (CNS). EAE animal models are widely studied as an animal model of human CNS demyelinating diseases, including the diseases MS and acute disseminated encephalomyelitis (ADEM). EAE is also the prototype for T-cell-mediated autoimmune disease in general. EAE can be induced using antigens in a number of species, including mice, rats, guinea pigs, rabbits and primates. The most commonly used antigens to induce EAE in rodents are spinal cord homogenate (SCH), purified myelin, myelin proteins such as MBP, PLP and MOG, or peptides of these proteins, all resulting in distinct models with different disease characteristics regarding both immunology and pathology. EAE also may be induced by the passive transfer of T cells specifically reactive to these myelin antigens. Depending on the antigen used and the genetic make-up of the animal, rodents can display a monophasic bout of EAE, a relapsing-remitting form, or chronic EAE. The typical susceptible rodent will debut with clinical symptoms around two weeks after immunization and present with a relapsing-remitting disease. The archetypical first clinical symptom is weakness of tail tonus that progresses to paralysis of the tail, followed by a progression up the body to affect the hind limbs and finally the forelimbs. However, similar to MS, the disease symptoms reflect the anatomical location of the inflammatory lesions, and may also include emotional liability, sensory loss, optic neuritis, difficulties with coordination and balance, and muscle weakness and spasms. Recovery from symptoms can be complete or partial and the time varies with symptoms and disease severity. Depending on the relapse-remission intervals, rats can have up to three bouts of disease within an experimental period.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

Amplification and Purification of pDNA

The plasmid construct encoding for rat interleukin-10 (pDNA-IL-10^{F129S}) has been previously described in detail in Milligan, et al., Pain 126(1-3): 294-308 (2006). In short, plasmid DNA comprising a bacterial backbone, a rat or human IL-10 sequence mutated in the hinge region flanked by ITR sequences, and selectable markers were produced. The pDNA-Control used herein was identical to the rat interleukin-10 plasmid construct except that a polyA sequence was substituted for the IL-10 encoding region (Milligan, et al., Pain 126(1-3): 294-308 (2006)), and purified using an endotoxin-free plasmid Giga purification kit (Qiagen). The endotoxin content of the pDNA-IL-10, as assessed by the photometric limulus amebocyte lysate (LAL) assay (BioWhittaker Inc.), was 0.0021±0.00034 ng LPS/μg DNA, a level that is generally considered negligible.

### Microparticle Preparation and Characterization

Microparticles were prepared using a modified double emulsion/solvent evaporation protocol (A. M. Tinsley-Bown, et al., J. of Controlled Release 66(2-3): 229-41 (2006)). Briefly, a 50:50 PLGA copolymer (MW 75,000, Lactel Absorbable Polymers) was dissolved in ethyl acetate (Sigma). Vehicle alone (phosphate buffered saline (PBS)+3% (w/v) sucrose (Sigma)) or pDNA in vehicle were emulsified in the PLGA solution followed by a second emulsion in a 5% (w/v) polyvinyl alcohol, 28% calcium chloride, 3% sucrose (Sigma) and 7% (v/v) ethyl acetate solution. After 4 hours of hardening in a wash solution, the resulting microparticles were collected, lyophilized and stored at 4° C. Scanning electron microscopy (SEM) was used to examine microparticle morphology. The diameters of >1000 microparticles present in 10 different images were measured with NIH ImageJ software and binned particle diameters were used to generate a normalized frequency distribution. The zeta potential of the microparticles was measured with a Nicomp 380 ZLS Zeta Potential Analyzer, and the endotoxin levels of the resultant microparticles were tested by the LAL assay, using serial dilution as a control for inhibition. The microparticles utilized exhibited a spherical and smooth morphology under SEM and a zeta-potential of −28.04±2.12 mV. The microparticles exhibited a heterogeneous size distribution with an overall median diameter of 4.67±0.26 μm, which is consistent with similar methods of microparticle manufacturing and the pDNA encapsulation efficiency for the particles was 55.1%.

Total pDNA encapsulation was assessed by extracting pDNA from microparticles via sodium hydroxide dissolution, measuring the absorbance at 260 nm and comparing obtained values to DNA standards at known concentrations. Final pDNA loadings were 8.78±0.65 μgpDNA/mgPLGA for PLGA-pDNA-IL-10 microparticles. Aqueous extraction of pDNA was conducted by dissolving microparticles in chloroform and allowing the pDNA to migrate into aqueous buffer. The extracted pDNA was subsequently concentrated by precipitation with ethanol and re-suspended in PBS+3% sucrose vehicle. The structural integrity of the aqueous extracted pDNA was compared against unencapsulated pDNA (which was similarly exposed to the aqueous extraction process) by loading 2 μg of total pDNA into the wells of a 1.0% agarose gel containing ethidium bromide, running the gel at 75 V for 2 hours, and imaging the gel with UV trans-illumination at 305 nm. Biological activity of aqueous extracted pDNA was assessed by lipofectamine-mediated transfection into human embryonic kindey-293 cells according to manufacturer protocols (Invitrogen) and IL-10 protein concentrations in cell culture supernatants collected 24 hours after transfection with aqueous extracted and unencapsulated pDNA were assessed by ELISA (R&D Systems). In vitro release profiling was conducted by incubating microparticles in PBS over time in a water bath at 37° C. and pDNA contents in the supernatant were quantified by a PicoGreen assay (Milligan, et al., Neuron Glia Biology 2(4) 293-308 (2006)).

Agarose gel electrophoresis of aqueous extracted pDNA from microparticles compared to unencapsulated pDNA indicated that a significant amount of the relaxed and supercoiled pDNA structural integrity was preserved after encapsulation, although a slight detection of linearized pDNA and slight alterations in the migration of multimeric pDNA species were observed after encapsulation. By comparing resultant IL-10 protein expression levels in the supernatants of human embryonic kidney-293 cells 24 hours after lipofectamine-mediated transfection with dose-matched microparticle extracted or unencapsulated pDNA, it was determined that the microparticle extracted pDNA-IL-10 exhibited a 96.8% biological activity retention for the resultant production of IL-10 (data not shown). In vitro pDNA release analysis demonstrated that 30% of the pDNA was released after 3 days and steady release was achieved for greater than 75 days. This two-phase release profile is a common characteristic of macromolecule release from emulsion based PLGA microparticles, where the enhanced phase of initial pDNA release is due to an increased pDNA content on or near the surface of the microparticles which is followed by a sustained release and diffusion of pDNA from the microparticle interior (Yeo, Archive of Endotoxin Res. 27(1): 1-12 (2004)). Endotoxin levels from microparticles with and without encapsulated pDNA were below the limits of detection for the LAL assay up to a microparticle concentration of 10 mg/ml (1 mg of microparticles/well).

Behavioral Testing in EAE Animal Models of MS Treated with Intrathecally Administered Unencapsulated or Microparticle Delivered pDNA-IL-10

Experimental autoimmune encephalomyelitis (EAE) is an animal model of multiple sclerosis induced by intradermal delivery of the myelin oligodendrocyte glycoprotein (MOG). A T-cell mounted attack on MOG is induced with symptom onset occurring 7-12 days after delivery. MOG was suspended in complete Freund's adjuvant and injected intradermally at the base of the tail under isoflurane anesthesia. Symptoms included motor weakness progressing to paralysis in a caudal to rostral distribution, and occurred in a relapsing remitting profile. Disease severity was monitored by measuring body weight and motor symptoms daily. In addition to motor scores, night time voluntary running wheel activity was measured. Wheel turns were monitored using the vitalview system allowing for remote testing. Wheel counts were summed over the 12 hour dark phase per night before MOG, after MOG, and after intrathecal delivery.

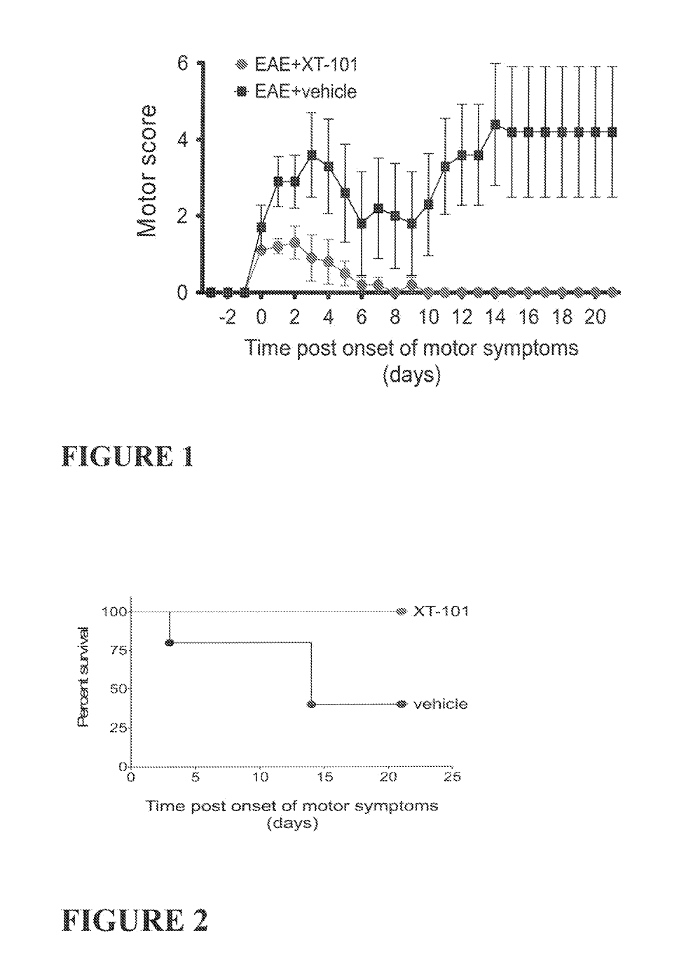

FIG. 1 is a graph plotting motor score versus time post-onset of motor symptoms (in days) in treated and untreated experimental autoimmune encephalomyelitis (EAE) rats. FIG. 1 demonstrates that EAE rats administered a single intrathecal injection of vehicle (squares) (PBS+3% (w/v) sucrose) sustain rapid decrease of motor score by day 4 post-onset, whereas EAE rats administered a single intrathecal injection of PLGA-pDNA-IL-10 (circles) after a decrease in motor score from days 4-10 regain and maintain a high motor score.

FIG. 2 demonstrates the remarkable effect of PLGA-pDNA-IL-10 administered on the survival of EAE rats. None of the rats receiving PLGA-pDNA-IL-10 produced severe enough symptoms to be euthanized, compared to approximately 65% of vehicle-injected rats.

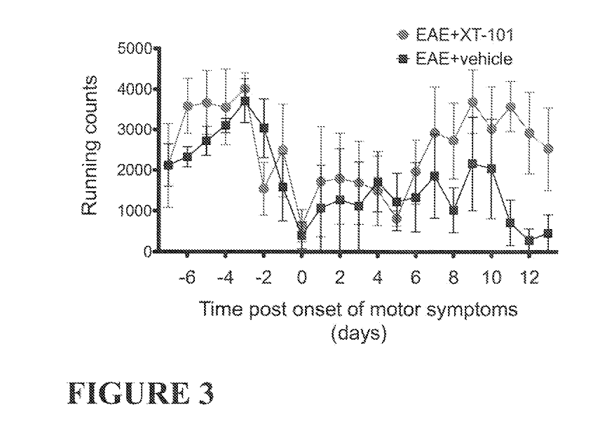

FIG. 3 shows the sharp decline in voluntary running the night before the onset of motor symptoms in EAE rats. However, the voluntary activity in rats following a single intrathecal injection of PLGA-pDNA-IL-10 returns to pre-symptom onset levels, where rats subjected to a single intrathecal injection of vehicle have a marked sustained decrease in running counts.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

We claim:

1. A method for treating multiple sclerosis or other demyelinating disorder, said method comprising administering to a subject a therapeutic microparticle composition comprising: plasmid DNA comprising a bacterial backbone and a nucleic acid sequence encoding interleukin-10; and microparticles encapsulating the plasmid DNA, wherein the microparticles degrade to deliver plasmid DNA over a period of greater than 40 days.

2. The method of claim 1, wherein the nucleic acid sequence encoding interleukin-10 has an amino acid substitution for wildtype phenylalanine at amino acid position 129.

3. The method of claim 2, wherein the amino acid substitution is selected from the group of serine, alanine, threonine or cysteine.

4. The method of claim 3, wherein the nucleic acid sequence encoding interleukin-10 encodes IL-10^{F129S}.

5. The method of claim 1, wherein the polymer comprises poly(lactic-co-glycolic acid).

6. The method of claim 1, wherein the therapeutic composition is administered intrathecally.

7. The method of claim 1, wherein the plasmid DNA comprises at least one nuclear targeting sequence 5′ to the one or more anti-inflammatory cytokine coding sequences.

8. The method of claim 1, wherein the therapeutic microparticle composition provides a therapeutically effective dose at about 0.010 μg pDNA per gram animal weight to about 0.90 μg pDNA per gram animal weight.

9. The method of claim 1, further comprising a diluent.

10. A method for treating symptoms of multiple sclerosis or other demyelinating disorders in a subject, said method comprising administering to a subject a therapeutic microparticle composition comprising: plasmid DNA comprising a bacterial backbone and at least one nucleic acid sequence encoding interleukin-10; and microparticles encapsulating the plasmid DNA, wherein the microparticles degrade to deliver plasmid DNA over a period of greater than 40 days, wherein the therapeutic microparticle composition provides a therapeutically effective dose at about 0.010 μg pDNA per gram animal weight to about 0.90 μg pDNA per gram animal weight.

11. The method of claim 10, wherein the nucleic acid sequence encoding interleukin-10 has an amino acid substitution for wildtype phenylalanine at amino acid position 129.

12. The method of claim 11, wherein the amino acid substitution is selected from the group of serine, alanine, threonine or cysteine.

13. The method of claim 12, wherein the nucleic acid sequence encoding interleukin-10 encodes IL-10^{F129S}.

14. The method of claim 10, wherein the polymer comprises poly(lactic-co-glycolic acid).

15. The method of claim 10, wherein the therapeutic composition is administered intrathecally.

16. The method of claim 10, wherein the plasmid DNA comprises at least one nuclear targeting sequence 5′ to the one or more anti-inflammatory cytokine coding sequences.

17. The method of claim 10, further comprising a diluent.

18. A method for preventing relapse of symptoms related to multiple sclerosis or other demyelinating disorders in a subject, said method comprising administering to a subject a therapeutic microparticle composition comprising: plasmid DNA comprising a bacterial backbone and at least one nucleic acid sequence encoding interleukin-10; microparticles encapsulating the plasmid DNA, wherein the microparticles degrade to deliver plasmid DNA over a period of greater than 40 days; and a diluent; wherein the therapeutic microparticle composition provides a therapeutically effective dose at about 0.010 μg pDNA per gram animal weight to about 0.90 μg pDNA per gram animal weight.

19. The method of claim 18, wherein the nucleic acid sequence encoding interleukin-10 has an amino acid substitution for wildtype phenylalanine at amino acid position 129.

20. The method of claim 19, wherein the amino acid substitution is selected from the group of serine, alanine, threonine or cysteine.

21. The method of claim 208, wherein the nucleic acid sequence encoding interleukin-10 encodes IL-10^{F129S}.

22. The method of claim 18, wherein the polymer comprises poly(lactic-co-glycolic acid).

23. The method of claim 18, wherein the therapeutic composition is administered intrathecally.

24. The method of claim 18, wherein the plasmid DNA comprises at least one nuclear targeting sequence 5′ to the one or more anti-inflammatory cytokine coding sequences.
